(19) United States
(12) Patent Application Publication
(10) Pub. No.: US 2023/0089767 A1
(43) Pub. Date: Mar. 23, 2023

(54) ENHANCING AUDITORY PERCEPTS WITH VESTIBULAR SIMULATION

(71) Applicant: Cochlear Limited, Macquarie University, NSW (AU)

(72) Inventor: Mattheus Johannes Petrus KILLIAN, Mechelen (BE)

(21) Appl. No.: 17/910,209

(22) PCT Filed: Feb. 16, 2021
(86) PCT No.: PCT/IB2021/051298

§ 371 Date: Sep. 8, 2022

Related U.S. Application Data
(60) Provisional application No. 62/992,280, filed on Mar. 20, 2020.

(51) Int. Cl. A61N 1/36; A61N 1/05

(52) U.S. Cl. CPC A61N 1/36038; A61N 1/0541

(57) ABSTRACT

Disclosed examples include technology to use residual vestibular function or residual vestibular nerve function of a recipient to provide low frequency information to enhance auditory percepts caused by auditory prostheses. An example can include adding a low frequency actuator to a cochlear implant for placement close to the vestibule of the ear. In addition or instead, one or more electrodes can be disposed proximate the vestibule of the ear to electrically stimulate residual vestibular function provided by, for example, the utricle and saccule. The stimulation provided by these components can be optimized for delivery to the vestibular system.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or sensory organ stimulation and/or lifestyle enhancement functions and/or recipient monitoring for a number of years.

The types of medical devices and the ranges of functions performed thereby have increased over the years. For example, many medical devices, sometimes referred to as “implantable medical devices”, now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an example, there is a method comprising: obtaining a sound signal; generating, based on the sound signal, cochlear stimulation configured to stimulate cochlear tissue; and generating, based on the sound signal, vestibular stimulation configured to stimulate vestibular tissue.

In another example, there is an apparatus, comprising a cochlear stimulator configured to stimulate cochlear tissue; a vestibular stimulator configured to stimulate vestibular tissue; and a sound processor. The sound processor is configured to generate cochlear stimulation with the cochlear stimulator based on a sound signal and generate vestibular stimulation with the vestibular stimulator based on the sound signal.

In yet another example, there is a method comprising: obtaining a sound signal; generating a vestibular-optimized sound signal based on the sound signal; and generating, based on the vestibular-optimized sound signal, vestibular stimulation configured to cause a hearing precept in a recipient.

### BRIEF DESCRIPTION OF THE DRAWINGS

The same number represents the same element or same type of element in all drawings.

FIG. 1 illustrates and example system for enhancing cochlear stimulation with vestibular stimulation.

FIG. 2 illustrates an example method for enhancing cochlear stimulation with vestibular stimulation.

FIG. 3 illustrates an example cochlear implant system that can benefit from use of the technologies disclosed herein.

FIG. 4 is a view of an example of a percutaneous bone conduction device that can benefit from use of the technologies disclosed herein.

FIG. 5 illustrates an example of a transcutaneous bone conduction device that can benefit from use of the technologies disclosed herein.

### DETAILED DESCRIPTION

Individuals affected by hearing loss can benefit from auditory prostheses. Such prostheses typically target cochlear tissue or the auditory nerve to cause recipients to experience hearing percepts. For example, the auditory prosthesis can receive a sound signal, process the signal, and stimulate cochlear tissue based thereon via, for example, electrical stimulation, bone conduction, or air-conduction. In some examples, the stimulation can be provided unilaterally or bilaterally. For instance, a recipient can have one stimulator disposed for stimulating a left side and another for stimulating a right side. In other examples, one stimulator can stimulate both left and right sides (e.g., a skull-mounted bone conductor configured to send signals bilaterally).

The sound signal on which the stimulation is based can include a variety of components. For example, the sound signal can include spectral content, which can be useful in sound segregation for a caused hearing percept. For instance, the spectral content can include a fundamental frequency that can be useful in speaker differentiation. Voiced speech of a typical adult male can have a fundamental frequency from 85 Hz to 180 Hz, and that of a typical adult female can range from 165 Hz to 255 Hz. Thus, being able to perceive a fundamental frequency can be useful in differentiating between typical male and female voices. Recipients of auditory prostheses can struggle to resolve fundamental frequency harmonic information. For example, some cochlear implants can have low spectral resolution at the level of the electrode but can obtain information on the harmonic frequency through the amplitude modulation in the presented spike trains. This information becomes locally available when multiple harmonics of the sound source fall within the band filters allocated to an electrode. Generally, such local availability of low frequency modulation based on a resonating sound source with a low fundamental frequency is applicable to higher frequencies but is not well represented at the lower frequency channels (where the actual fundamental frequency is located) when multiple low frequency harmonics do not fall into the band filter allocated to a lower frequency electrode of an electrically-stimulating cochlear implant. Specific information in the cochlear apical low frequency region within the range of the fundamental of a speaker is generally poorly resolved with cochlear implants and most implant users have difficulty with identification of a speaker. In principle, amplitude modulation at the fundamental frequency of a sound source can become apparent in all cochlear implant channels. Resonating sound sources with low frequency fundamentals generally create modulation over the whole electrode array (particularly at higher frequency channels), while sound sources with high fundamental frequencies create high frequency modulations at the higher frequency channels. Cochlear implant recipients generally can detect low modulation frequencies (e.g., up to 300-500 Hz) but can have difficulty in detecting higher frequency modulations.

Disclosed examples include technology that can be used to convey such low frequency acoustic information (among other information) via stimulation of vestibular tissue. For example, disclosed examples can be used to stimulate a vestibular organ to convey fundamental frequency information. Research suggests that residual hearing can exist in the vestibular system rather than simply the cochlea. See, e.g., Emami and Daneshi, “Vestibular Hearing and Neural Synchronization”, International Scholarly Research Network Otolaryngology, doi:10.5402/2012/246065 (2012), which is hereby incorporated by reference in its entirety for any and all purposes.

Disclosed examples include technology to use residual vestibular function of a recipient to provide low frequency information to enhance auditory percepts caused by auditory prostheses. Such stimulation may also positively affect the recipient's sense of balance. Example enhancements include causing percepts beneficial for identification of sound sources with vestibular stimulation. For example, disclosed technology can provide vestibular stimulation to target residual vestibular function based on low frequency components of the sound signal to enhance the low frequency input provided by a cochlear implant that may be obscured due to low resolution at low-frequencies. The fundamental frequency can be identified in the signal and used to stimulate the vestibular system. In addition or instead, the stimulation can be based on low frequency multiples of the detected fundamental in the sound spectrum or transposed fundamentals (e.g., ½, ⅓, or ¼ of the fundamental frequency) can be enhanced and delivered via vestibular stimulation.

The stimulation can be provided to the vestibular system via a dedicated vestibular stimulator. The vestibular stimulator can be a low frequency actuator added to a cochlear implant for placement proximate the vestibule of the ear (e.g., rather than proximate the apex of the cochlea). In addition or instead, the vestibular stimulator can be or include one or more electrodes disposed proximate the vestibule of the ear to electrically stimulate residual vestibular function (e.g., provided by the utricle and saccule). The electrical stimulation can have pulse rates or a modulating high frequency pulse train aligned to the fundamental frequency of an incoming sound to induce vestibular hearing. Yet another example of a vestibular stimulator is an air-conduction based stimulation, such as by generating air-conducted sound waves with a speaker and directing such sound waves down an ear canal of the recipient. An example technique can be integrated into a contralateral hearing aid for a recipient having little to no low frequency hearing but still some low frequency acoustical function available in the contralateral ear that can result in activation of vestibular tissue.

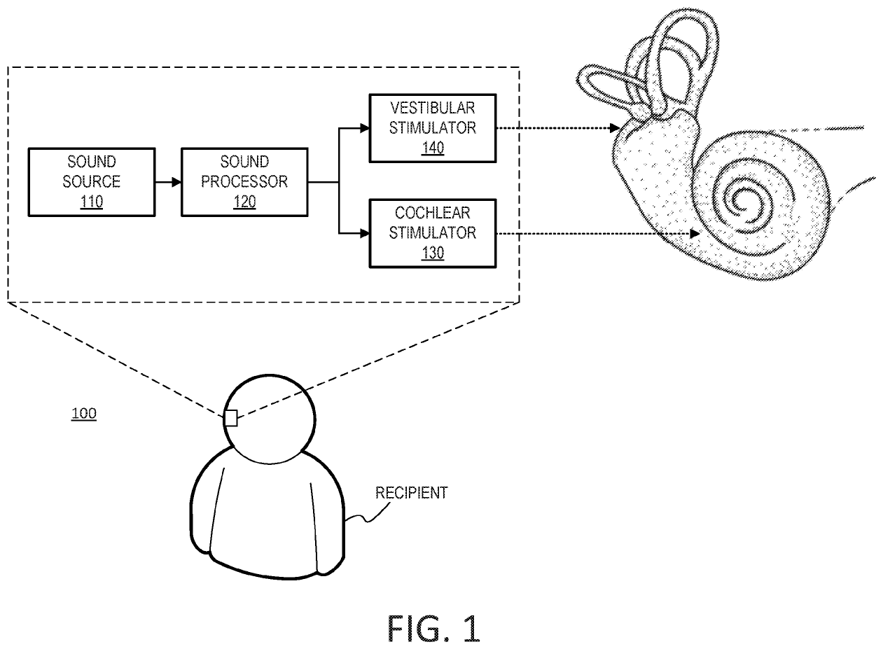

An example system that implements an example of such a vestibular stimulation technique is described in relation to FIG. 1.

### System

FIG. 1 illustrates and example system 100 for enhancing cochlear stimulation with vestibular stimulation. As illustrated, the system can include a sound source 110, a sound processor 120, a cochlear stimulator 130, and a vestibular stimulator 140. These components can be arranged in one or more housings. For example, the system 100 can include a wearable housing configured to be worn by a recipient in addition to or instead of an implantable housing configured to be disposed beneath the skin of the recipient. The cochlear stimulator 130 and the vestibular stimulator 140 can be disposed within or extend from one or more of such housings. The sound source 110 and the sound processor 120 can also be disposed in one or more of such housings. In an example, the component are disposed in an implantable housing and act as a totally-implantable cochlear implant having vestibular stimulation capability.

The sound source 110 can be a component of the system 100 configured to detect or receive a sound and generate a signal (e.g., an electrical signal representative of the detected sound) based thereon. The sound source 110 can include one or more microphones that produce a signal based on sounds in the environment around the microphone. The sound source 110 can include a remote device connected via a wireless connection. Such a wireless connection can include an FM (Frequency Modulation) connection, such as can be used to communicate with a remote microphone (e.g., a COCHLEAR TRUE WIRELESS MINI MICROPHONE2+), a television audio streaming device, or a phone clip device, among other devices having FM transmission capabilities. The wireless connection can also include a radiofrequency connection, such as via a BLUETOOTH connection to a wireless streaming device. The system can be arranged such that the environmental sound related signal produced by the sound source 110 is provided as input into the sound processor 120.

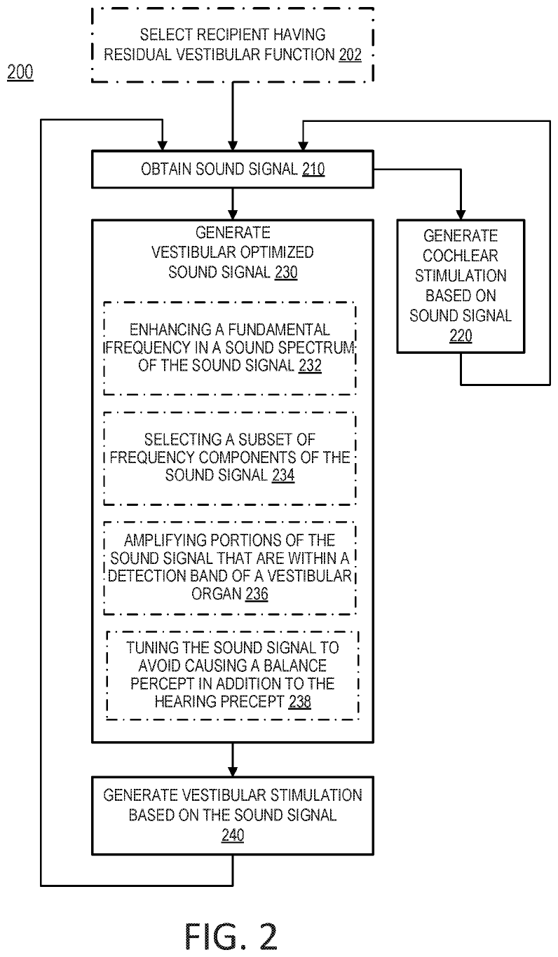

The sound processor 120 can be a component configured to process sound signals from the sound source 110 and cause stimulation based thereon. The sound processor 120 can obtain the sound signal, process the sound signal, and produce one or more output signals based thereon. The one or more output signals can be provided to the cochlear stimulator 130 and the vestibular stimulator 140 to cause stimulation. For example, the sound processor 120 can be configured to produce a cochlear optimized output signal that is provided to the cochlear stimulator 130, which causes cochlear stimulation based on the cochlear stimulation output signal. Likewise, the sound processor 120 can be configured to produce a vestibular optimized output signal that is provided to the vestibular stimulator 140, which causes vestibular stimulation based on the vestibular stimulation output signal. The sound processor 120 can be configured to perform one or more operations, such as those described in relation to method 200 of FIG. 2, including generating an optimized sound signal for providing stimulation via the vestibular stimulator 140 as described in relation to operation 230.

The processing performed by the sound processor 120 can include the implementation of one more sound processing strategies to process the sound signal. An example process for modifying a sound signal that can be implemented by the sound processor 120 is provided in U.S. Pat. No. 7,561,709, issued Jul. 14, 2009, which is hereby incorporated by reference in its entirety for any and all purposes. For example, the sound processor 120 can selectively increase a modulation depth of certain channels of the sound signal based on a stimulation strategy.

Among various components, the sound processor 120 can include banks of filters and templates to process the sound signal and produce an output on which stimulation can be provided. In an example, the components can include fundamental frequency templates to isolate or otherwise separate a fundamental frequency or multiples thereof from the sound signal. An example for determining a fundamental frequency for use in stimulation herein can be as described in U.S. Pat. No. 7,231,257, issued Jun. 12, 2007, which is hereby incorporated by reference in its entirety for any and all purposes. The resulting signals can used as the basis for further processing or used for stimulation. For example, the sound processor 120 can isolate the fundamental frequency and cause the vestibular stimulator 140 to provide stimulation based thereon.

As another example, the sound processor 120 can include a fundamental frequency estimator, which is described in U.S. Pat. No. 9,084,893, issued Jul. 21, 2015, which is hereby incorporated by reference in its entirety for any and all purposes. The fundamental frequency estimator can include, for example, a phase-vocoder fast Fourier transform filterbank that processes the electrical signal to provide an estimate of the frequency and power of any sinusoidal frequency components present in the electrical signal.

As described above, the sound processor 120 can produce separate cochlear stimulation and vestibular stimulation outputs. Such outputs can be based on a same or separate sound inputs and can share one or more aspects of a sound processing path though the sound processor. In many examples, the sound processor 120 includes a cochlear stimulation branch for producing the cochlear stimulation output optimized for stimulating cochlear tissue and a vestibular stimulation branch for producing vestibular stimulation output optimized for stimulating vestibular tissue. In an implementation, the sound processor 120 can extract a subset of the sound signal (e.g., corresponding to a fundamental frequency or another low frequency component of a sound signal) and pass the extracted frequency through a vestibular stimulation sound path for producing a vestibular stimulation output. The remainder or entirety of the sound signal can be passed through a cochlear stimulation branch to produce a cochlear stimulation output. The cochlear stimulation output and the vestibular stimulation output can then be provided to the cochlear stimulator 130 and the vestibular stimulator 140, respectively.

The cochlear stimulator 130 can be a component of the system 100 configured to stimulate cochlear tissue. The cochlear stimulator 130 can receive the cochlear stimulation output from the sound processor 120 and cause stimulation based thereon. The cochlear stimulator 130 can take any of a variety of forms. In one example, the cochlear stimulator 130 comprises one or more electrodes for providing electrical stimulation to cochlear tissue. The electrodes can be part of a stimulation array of the cochlear stimulator configured to be disposed in a cochlea of the recipient. In another example, the cochlear stimulator 130 is a bone conduction component. For instance, the cochlear stimulator 130 can include an implantable or wearable vibratory actuator tuned to generate vibrations to travel through bone to vibrationally stimulate cochlear tissue. In yet another example, the cochlear stimulator 130 is an air-conduction component configured to stimulate a cochlea with vibrations conducted via air though the ear canal. Other cochlear stimulation techniques or combinations thereof can be used.

The vestibular stimulator 140 can be a component of the system 100 configured to stimulate vestibular tissue. The vestibular stimulator 140 can receive the vestibular stimulation output from the sound processor 120 and cause stimulation based thereon. For example, the vestibular stimulator 140 can be configured to be disposed proximate an otolith organ of the vestibular system. The vestibular stimulator 140 can be configured to be disposed proximate the oval window of the recipient for providing stimulation through the oval window to target vestibular tissue. The vestibular stimulator 140 can take any of a variety of forms. In one example, the vestibular stimulator 140 comprises one or more electrodes. The electrodes can be one or more extracochlear electrodes (i.e., electrodes disposed outside of the recipient's cochlea). In some examples, the electrodes can be one or more intravestibular electrodes configured to be disposed in vestibular tissue. In some examples, the electrodes can be intracochlear electrodes (e.g., disposed on an intracochlear array to be situated in the basal part of the cochlea). For example, the intracochlear electrodes can be configured to stimulate the saccule, which is connected to the cochlea via the ductus reuniens. The vestibular stimulator 140 can be configured to stimulate vestibular tissue by, for example, being sized and shaped to be disposed proximate vestibular tissue to provide stimulation to particular vestibular tissue. In some examples, the vestibular stimulator 140 is an air-conduction component configured to stimulate the vestibular system with vibrations conducted via air. In some examples, the vestibular stimulator 140 is a bone conduction component. For instance, the vestibular stimulator 140 can include an implantable or wearable vibratory actuator tuned to generate vibrations to travel through bone to vibrationally stimulate vestibular tissue. Other kinds of stimulation or combinations thereof can be used for vestibular stimulation.

Additional example implementations of a vestibular stimulator 140 that can be used to provide stimulation that can be used to stimulate the recipient's vestibular system are described in relation to European Patent Application No. 19382629.4 and European Patent Application No. 19382632.8, both of which were filed on Jul. 24, 2019, and are hereby incorporated by reference in their entirety for any and all purposes.

As described above, both the vestibular stimulator 140 and the cochlear stimulator 130 can used bone-conduction, air conduction, electrical stimulation, other techniques, or combinations thereof for stimulating respective tissue. Though the stimulators 130, 140 can use similar stimulation modalities, each can be configured for stimulating their respective kinds of tissue. As an example, the stimulators 130, 140 can be optimized to produce frequencies in particular ranges. For instance, the cochlear stimulator 130 can be tuned to produce frequencies in the range of typical human hearing (e.g., 20 Hz to 20 KHz), while the vestibular stimulator 140 can be tuned to can cause stimulation at relatively lower frequencies, such as at or below approximately 900 Hz, 800 Hz, or 500 Hz. There can be overlap between the frequencies to which the cochlear stimulator 130 and the vestibular stimulator 140 are tuned. In addition, the stimulators 130, 140 can be configured to produce stimulation at such frequencies at a sufficiently high intensity to cause a particular kind of percept (e.g., a detectable percept). The level can be, for example, at least 90 dB. The target frequency can target a fundamental frequency. As another example, the stimulators 130, 140 can be sized and shaped to be disposed in therapeutic proximity to target tissue to be stimulated. For instance, the cochlear stimulator 130 can include an electrode assembly having electrodes disposed thereon, and such components can be sized and shaped for insertion and retention in a recipient's cochlear. The vestibular stimulator 140 can be sized and shaped in such a matter to be placed therapeutically proximate to the vestibular tissue and deliver stimulation to vestibular tissue.

The components of the system 100 can be used to implement a method for enhancing cochlear stimulation with vestibular stimulation. An example of such a method is described in conjunction with FIG. 2.

### Method

FIG. 2 illustrates a method 200 for enhancing cochlear stimulation with vestibular stimulation. The method 200 can begin with operation 210 or, in some implementations, operation 202.

Operation 202 can include selecting a recipient having residual vestibular function. For example, a recipient can be tested for vestibular function. The vestibular function can be vestibular balance function or vestibular hearing function. The vestibular function can be tested by applying stimulation to the recipient's vestibular system and determining whether the recipient indicates that a vestibular percept was detected. In some examples, the testing can further include determining a range of frequencies for which the recipient's vestibular system perceives stimulation as auditory signals. Such a range can be used to, for example, select a subset frequencies in operation 234 and can be used as part of operation 236 to amplify portions of a sound signal that are within a detection band of a vestibular organ. In some examples, the recipient may lack vestibular function, but the vestibular nerve can still respond to electrical stimulation. Such residual vestibular nerve function can be tested with, for example, electrical stimulation rather than acoustic or vibratory stimulation. In such examples, the method can include selecting a recipient having residual vestibular nerve function. Following operation 202, the flow of the method 200 can move to operation 210.

Operation 210 can include obtaining a sound signal. For example, the sound signal 210 can be obtained by or from the sound source 110. The sound signal can be an analog or electrical signal indicative of sound. Where there are multiple sound sources 110, the output of the multiple sound sources can be mixed or otherwise combined together to form a single sound signal. The sound signal can be obtained by, for example, a wearable auditory device. The sound signal can include a set of frequency components. Following operation 210, the flow of the method 200 can move to operations 220 and 230.

Operation 220 can include generating, based on the sound signal, cochlear stimulation configured to stimulate cochlear tissue. In an example, the sound processor 120 generates a cochlear optimized output signal based on the sound signal. The generating of the cochlear optimized output signal can include optimizing the sound signal for delivery via the cochlear stimulator. Example sound processing techniques that can be used as part of this process include applying one or more sound processing operations based on one or more sound processing strategies, such as are described in more detail in relation to the sound processor 120, above. Additional example sound processing operations can include, for example gain adjustments (e.g., multichannel gain control), noise reduction operations, or signal enhancement operations (e.g., speech enhancement, wind reduction), other operations, or combinations thereof, in one or more of the channels. Noise reduction can include processing operations that identify unwanted (noise) components of a signal, and then subsequently reduce the presence of these unwanted components. Signal enhancement can refer to processing operations that identify the target signals (e.g., speech or music) and then subsequently increase the presence of these target signal components. Speech enhancement is a particular type of signal enhancement. Additional sound processing operations that can be performed include adaptive dynamic range optimization operations, automatic gain control operations, channel combiner operations, mixing operations, fast Fourier transform operations, level detection operations, beamforming operations, windowing operations, calibration filtering operations, pre-emphasis operations, other operations, and combinations thereof. Additional examples of operations and techniques that can be used to modify the sound signal and generate cochlear stimulation based thereon are described in U.S. Pat. Nos. 9,473,852 and 9,338,567, which are both incorporated herein by reference for any and all purposes.

The cochlear stimulator 130 can provide stimulation based on the cochlear stimulation output signal. The cochlear stimulation can be generated by, for example, an implanted or external (e.g., wearable) medical device. The cochlear stimulation can take any of a variety of forms, such as electrical stimulation or vibratory stimulation (e.g., bone-conduction or air-conduction vibratory stimulation). For example, where the cochlear stimulator 130 includes an electrical stimulator, a pulse generator of the cochlear stimulator 130 can generate one or more electrical pulses based on the cochlear optimized output signal and the pulses are delivered via one or more electrodes to cochlear tissue to cause hearing percepts in the recipient. Where the cochlear stimulator includes a vibratory stimulator, the vibratory stimulator can be made to vibrate based on the cochlear stimulation output signals. The vibrations can be conducted (e.g., via one or both of air and bone) to cochlear tissue to cause the tissue to move, thereby causing the recipient to experience cochlear hearing percept. Other techniques and combinations thereof can be used.

Following operation 220, the flow of the method 200 can return to operation 210.

Operation 230 can include generating, based on the sound signal, a vestibular optimized sound signal. This operation can include optimizing the sound signal for delivery to target the vestibular tissue. The sound signal can include components that can be optimized or isolated for use in generating the vestibular optimized sound signal. For instance, one or more low frequency components of the sound signal can be optimized. For example, the vestibular optimized sound signal can be generated to convey a fundamental frequency of the sound signal rather than the multiples of the fundamental frequency.

In examples, operation 230 can include one or more of operation 232, 234, 236, and 238.

Operation 232 can include enhancing a fundamental frequency in a sound spectrum of the sound signal. Enhancing the fundamental frequency can include boosting an intensity of the fundamental frequency relative to other signals in the sound signal. Such enhancing can include removing non-fundamental frequency components of the sound signal or increasing the intensity of the signal at the fundamental frequency. In some examples one or more fundamental frequency templates are applied to isolate or otherwise identify the fundamental frequency from the sound signal. In some examples estimating the fundamental frequency is performed using a fundamental frequency estimator. Then a new sound single can be generated using the estimated fundamental frequency or signals corresponding the estimated fundamental frequency can be enhanced relative to other signals. In some examples, the multiples of the fundamental frequency (harmonics) can be determined from a spectrogram. When multiple harmonics of a sound source pass through one filter, then beats can be detectable in the output at the fundamental frequency. So isolation of the fundamental frequency can be used to provide vibratory or electrical stimulation at the fundamental frequency. Another approach can include detecting beat (modulation) frequency in a filter output and deduce the fundamental frequency based thereon. Recipients can detect fundamental frequency via cochlear stimulation through the modulation in the electrical stimulation pattern that corresponds to the rate or modulation of the electrical pulse train in the vestibular system, which can enhance a quality of the resulting sound percept and give the recipient input to perceive the sound as coming from a single source. Where there are multiple sound origins in an environment, the modulation/beats can be more complex, which can provide information that multiple sound sources are actively picked up by the sound source 110.

Operation 234 can include identifying a subset of frequency components of the sound signal. The resulting vestibular-optimized sound signal can be processed to include the subset and lack frequency components outside the subset. For example, the subset can include components of the sound signal that are below a particular threshold. For instance, the threshold can represent a frequency below which components of the sound signal are designated for use in vestibular stimulation and above or equal to which the components of the sound signal designated for use in cochlear stimulation. Example thresholds can be less than or equal to approximately 900 Hz, 800 Hz, or 500 Hz.

Operation 236 can include amplifying portions of the sound signal that are within a detection band of a vestibular organ. In some examples, this operation 236 can include determining a detection band of the vestibular organ. In some examples, the detection band includes the subset of frequency components described above in operation 234. In some examples, the detection band is customized to the recipient (e.g., as in operation 202), is a detection band believed to be present for the recipient, or is a detection band typical for people of a particular demographic (e.g., individuals having a same conduction).

Operation 238 can include tuning the sound signal to avoid causing a deleterious balance percept in addition to the hearing percept. Tuning the sound signal can include, for example, selecting sound signal characteristics such that when vestibular stimulation is provided based on the sound output, the vestibular stimulation is configured to promote a hearing percept without causing a deleterious balance percept. This can include tuning the sound signal to avoid causing a balance percept at all or to avoid causing an undesirable or unintended balance percept. The tuning can include for example, selecting particular frequencies, intensities, other characteristics, or combinations thereof.

In some examples, operation 238 can include tuning the sound signal to cause a beneficial balance percept. This beneficial balance percept caused by the tuned sound signal can be in addition to or instead of the hearing percept caused by the sound signal. In an example, an extracochlear reference electrode can be positioned in such a way that an intracochlear stimulation path passes along the vestibular nerve. A reference electrode (e.g. a ball electrode) can be placed on or through the footplate of the stapes (e.g., oval window). The low frequency stimulus (e.g., low pulse rate or low frequency modulated high pulse rate) can then be provided via one or more basal intracochlear electrodes with reference to the ball electrode to focus the low frequency stimulation on the vestibular nerve. Such a signal can be tuned to accentuate balance precepts provided primarily via the vestibular stimulator 140. This stimulation can be in addition to or instead of providing such stimuli via the apical intracochlear channels.

Following operation 230, the flow of the method 200 can move to operation 240.

Operation 240 can include generating, based on the sound signal, vestibular stimulation configured to stimulate vestibular tissue. The vestibular stimulation can be provided by, for example, an implanted medical device. Generating the vestibular stimulation can include generating vibratory stimulation (e.g., when the vestibular system is at least partially intact) or electrical stimulation (e.g., when the vestibular system is non-functional but the neural tissue is still intact). Generating the electrical stimulation can include generating the stimulation via one or more vestibular electrodes disposed proximate an otolith organ. Generating the vibratory stimulation can include generating the stimulation via a vibratory actuator disposed proximate the otolith organ. In some examples, the vestibular stimulation and the cochlear stimulation can be based on a same subset of the set of frequency components of the sound input. Where the method 200 includes operation 230, the generating of the vestibular stimulation can be based on the vestibular optimized sound signal, thereby being based on the sound signal. As described above, the vestibular stimulator 140 can provide stimulation in any of a variety of ways, such as via electrical stimulation or vibratory stimulation. In some examples, combinations of stimulation modalities can be used depending in the implementation of the system 100 performing the method 200. The vestibular stimulation of the vestibular tissue can be delivered substantially simultaneously with the cochlear stimulation of the cochlear tissue. For example, the substantial simultaneity of the stimulation can result in the stimulation being perceived as being part of a same sound event. In an example, the recipient's cochlea can only detect low frequency electrical pulse train modulations. In this and other examples, modulated pulse trains (e.g. carrier frequency of 1000 pulses per second) can be provided in the range of detectable modulations (e.g., around 300) or a pulse train at that low rate.

In examples, the vestibular stimulation can be not only to cause or enhance an auditory percept, but also cause (or be in addition to stimulation that causes) a vestibular balance percept are both optimized. For example, accelerometer or other data can be used to determine and generate a vestibular stimulation signal to cause a balance percept to be experienced by the recipient. Thus, in at least some examples, both acoustic input (e.g., from a sound source) and balance input (e.g., from an accelerometer or other sensor) can be used to cause vestibular simulation. In further examples, vestibular stimulation can be not only to cause or enhance an auditory percept, but also cause (or be in addition to stimulation that causes) masking of vestibular noise signals generated by the recipient's peripheral vestibular system (e.g., thereby preventing erroneous balance information generated by the peripheral vestibular system from being sent to the brain of the recipient), such as is disclosed in the subject matter previously incorporated by reference herein.

Following operation 240, the flow of the method 200 can return to operation 210.

### Example Implementations

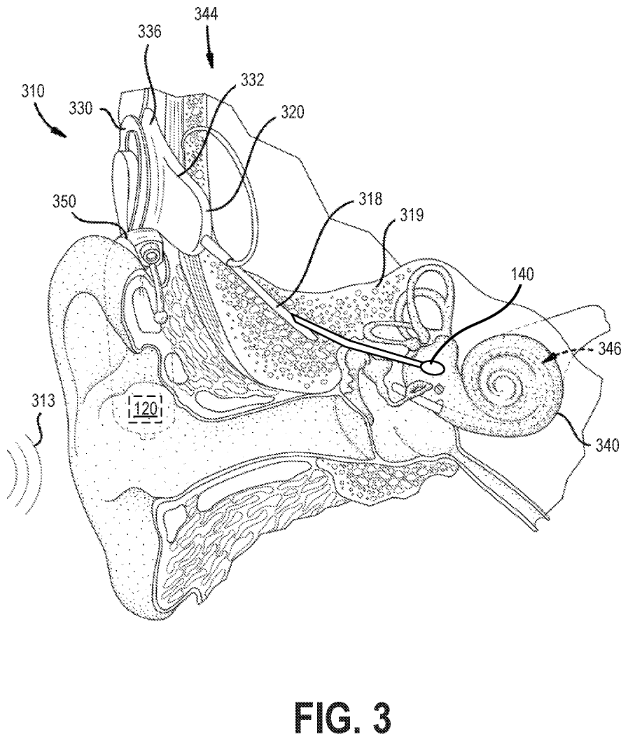

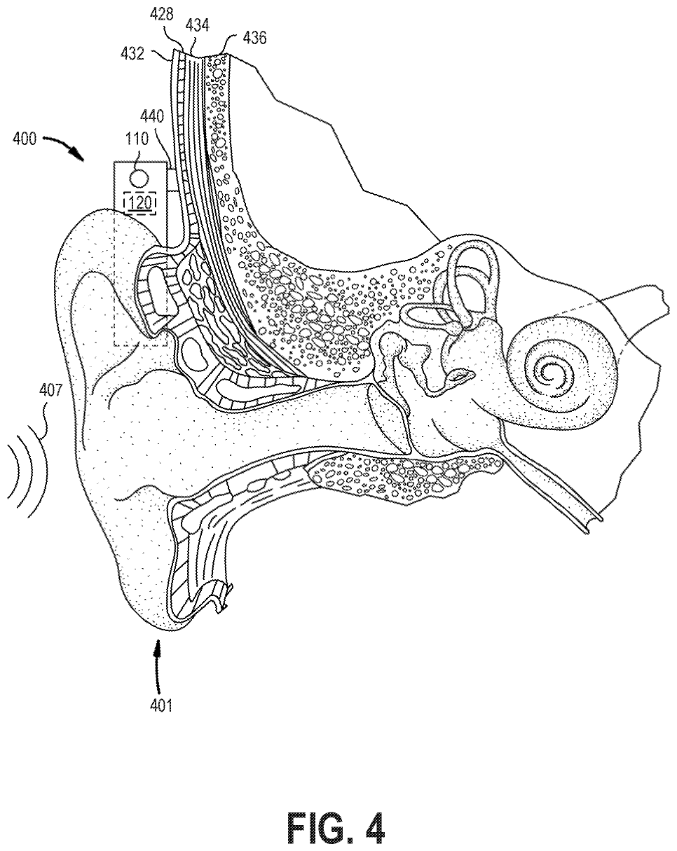

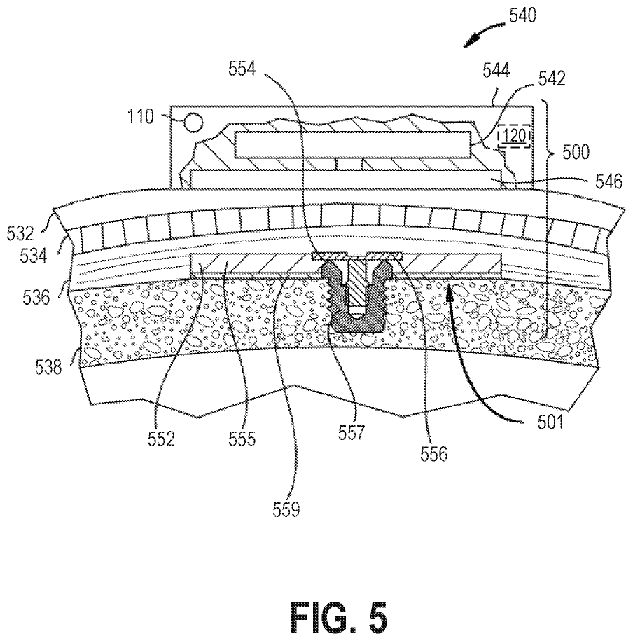

Vestibular stimulation can enhance audio percepts caused by a variety of different auditory prostheses. Example auditory prostheses can include a cochlear implant, an electroacoustic device, a percutaneous bone conduction device, a passive transcutaneous bone conduction device, an active transcutaneous bone conduction device, a middle ear device, a totally-implantable auditory device, a tinnitus management device, a mostly-implantable auditory device, an auditory brainstem implant device, a hearing aid, a tooth-anchored hearing device, a personal sound amplification product, other auditory prostheses, and combinations of the foregoing (e.g., binaural systems that include a prosthesis for a first ear of a recipient and a prosthesis of a same or different type for the second ear). Examples of such prostheses are described in more detail in FIGS. 3-5, below. For example, vestibular stimulation to cause hearing percepts can be added to a cochlear implant system 310 as described in FIG. 3, a percutaneous bone conduction device 400 as described in FIG. 4, or a transcutaneous bone conduction device 500 as described in FIG. 5. These different sensory prostheses can benefit from use with the systems and processes described above. In examples, a bone conduction device can be configured to deliver stimulation unilaterally (e.g., the bone conduction device can be disposed proximate a vestibular organ and be configured to focus stimulation unilaterally) or bilaterally (e.g., a bone conductor on the skull can be configured to send stimulation bilaterally).

### Example Cochlear Implant

FIG. 3 illustrates an example cochlear implant system 310 that can benefit from use of the technologies disclosed herein. The cochlear implant system 310 includes an implantable component 344 having an internal receiver/transceiver unit 332, a stimulator unit 320, and the cochlear stimulator 130 in the form of an elongate lead 318. The illustrated cochlear implant system 310 further includes the vestibular stimulator 140. The vestibular stimulator 140 is illustrated as being an electrical or vibratory stimulator disposed proximate the recipient's vestibular tissue. The vestibular stimulator 140 can be disposed proximate the semicircular canals, otolith organs, vestibular nerve, or in other locations. The internal receiver/transceiver unit 332 permits the cochlear implant system 310 to receive signals from and/or transmit signals to an external device 350. The external device 350 can be a head-worn device that includes the sound processor 120, the sound source 110, and a receiver/transceiver coil 330, among other components.

The implantable component 344 includes an internal coil 336 and a magnet fixed relative to the internal coil 336. The magnet can be embedded in a biocompatible encapsulant, along with the internal coil 336. Signals sent generally correspond to external sound 313. The internal receiver/transceiver unit 332 and the stimulator unit 320 are hermetically sealed within a biocompatible housing, sometimes collectively referred to as a stimulator/receiver unit. Included magnets can facilitate the operational alignment of an external coil 330 and the internal coil 336, enabling the internal coil 336 to receive power and stimulation data from the external coil 330. The external coil 330 is contained within an external portion. The stimulator unit 320 can include or be connected to the sound processor 120. The elongate lead 318 has a proximal end connected to the stimulator unit 320, and a distal end 346 implanted in a cochlea 340 of the recipient. The elongate lead 318 extends from stimulator unit 320 to the cochlea 340 through a mastoid bone 319 of the recipient. The elongate lead 318 is used to provide electrical stimulation to the cochlea 340 based on the stimulation data. Stimulation data can also be created for the vestibular stimulator 140 using the stimulator unit 320. Stimulation can be provided via an electrode for electrical stimulation or an actuator for low frequency vibratory stimulation. For electrical vestibular stimulation, the electrical vestibular stimulator and/or components of the electrical cochlear stimulator can be used. The stimulation data for both the elongate lead 318 (corresponding to the cochlear stimulator 130) and for the vestibular stimulator 140 can be created based on a same external sound 313 using the sound processor 120 and the stimulator unit 320.

In certain examples, the external coil 330 transmits electrical signals (e.g., power and stimulation data) to the internal coil 336 via a radio frequency (RF) link. The internal coil 336 is typically a wire antenna coil having multiple turns of electrically insulated single-strand or multi-strand platinum or gold wire. The electrical insulation of the internal coil 336 can be provided by a flexible silicone molding. Various types of energy transfer, such as infrared (IR), electromagnetic, capacitive and inductive transfer, can be used to transfer the power and/or data from external device to cochlear implant. While the above description has described internal and external coils being formed from insulated wire, in many cases, the internal and/or external coils can be implemented via electrically conductive traces.

### Percutaneous Bone Conduction Device

FIG. 4 is a view of an example of a percutaneous bone conduction device 400 that can benefit from use of the technologies disclosed herein. For example, sensory percepts caused by the percutaneous bone conduction device 400 can be enhanced by vestibular stimulation using one or more of the techniques described herein. The bone conduction device 400 is positioned behind an outer ear of a recipient of the device. The bone conduction device 400 includes a sound source 110 to receive sound signals 407. The sound source 110 can be, for example, a microphone or telecoil. In the present example, the sound source 110 can be located, for example, on or in the bone conduction device 400, or on a cable extending from the bone conduction device 400. The bone conduction device 400 further includes the sound processor 120, a vibrating actuator and various other operational components.

The sound source 110 can convert received sound signals into electrical signals. These electrical signals are processed by the sound processor 120. The sound processor 120 generates control signals that cause the actuator to vibrate. The actuator converts the electrical signals into mechanical force to impart vibrations to a skull bone 436 of the recipient to cause motion of the cochlear fluid, thereby stimulating the cochlea and resulting in the perception of the received sound signals.

In some implementations, the percutaneous bone conduction device 400 can have a vibratory actuator that acts as both the cochlear stimulator 130 and the vestibular stimulator 140. For example the actuator can generate vibrations configured to stimulate the cochlea as well as vibrations configured to stimulate the recipient's vestibular system (e.g., by having vibration frequencies selected to stimulate the vestibular system). In addition or instead, there can be a separate component (e.g., separate vibratory actuator or implantable electrical stimulator).

The bone conduction device 400 further includes a coupling apparatus 440 to attach the bone conduction device 400 to the recipient. In the illustrated example, the coupling apparatus 440 is attached to an anchor system implanted in the recipient. An exemplary anchor system (also referred to as a fixation system) may include a percutaneous abutment fixed to the skull bone 436. The abutment extends from the skull bone 436 through muscle 434, fat 428 and skin 432 so that the coupling apparatus 440 may be attached thereto. Such a percutaneous abutment provides an attachment location for the coupling apparatus 440 that facilitates efficient transmission of mechanical force.

### Transcutaneous Bone Conduction Device

FIG. 5 illustrates an example of a transcutaneous bone conduction device 500 having a passive implantable component 501 that can benefit from use of the technologies disclosed herein. The transcutaneous bone conduction device includes an external device 540 and an optional implantable component 501. The implantable component 501 includes a passive plate 555 mounted on the bone 538 and is transcutaneously coupled with a cochlear stimulator 130 in the form of a vibrating actuator 542 located in a housing 544 of the external device 540. The plate 555 may be in the form of a permanent magnet or in another form that generates or is reactive to a magnetic field, or otherwise permits the establishment of magnetic attraction between the external device 540 and the implantable component 550 sufficient to hold the external device 540 against the skin 532 of the recipient.

In some implementations, the vibrating actuator 542 can act as both the cochlear stimulator 130 and the vestibular stimulator 140. For example the vibrating actuator 542 can generate vibrations configured to stimulate the cochlea as well as vibrations configured to stimulate the recipient's vestibular system (e.g., by having vibration frequencies selected to stimulate the vestibular system). In addition or instead, there can be a separate component (e.g., separate vibratory actuator or implantable electrical stimulator).

In an example, the vibrating actuator 542 is a component that converts electrical signals into vibration. In operation, sound source 110 converts sound into electrical signals. The transcutaneous bone conduction device 500 can provide these electrical signals directly to the vibrating actuator 542 or to the sound processor 120 that processes the electrical signals, and then provides those processed signals to a vibrating actuator 542. The vibrating actuator 542 converts the electrical signals (processed or unprocessed) into vibrations. Because the vibrating actuator 542 is mechanically coupled to a plate 546, the vibrations are transferred from the vibrating actuator 542 to the plate 546. An implanted plate assembly 552 is part of the implantable component 550, and is made of a ferromagnetic material that may be in the form of a permanent magnet, that generates and/or is reactive to a magnetic field, or otherwise permits the establishment of a magnetic attraction between the external device 540 and the implantable component 550 sufficient to hold the external device 540 against the skin 532 of the recipient. Accordingly, vibrations produced by the vibrating actuator 542 of the external device 540 are transferred from plate 546 across the skin 532, fat 534, and muscle 536 to the plate 555 of the plate assembly 552. This may be accomplished as a result of mechanical conduction of the vibrations through the tissue, resulting from the external device 540 being in direct contact with the skin 532 and/or from the magnetic field between the two plates 546, 555. These vibrations are transferred without penetrating the skin 532 with a solid object such as an abutment.

As may be seen, the implanted plate assembly 552 is substantially rigidly attached to a bone fixture 557 in this example. But other bone fixtures may be used instead in this and other examples. In this regard, the implantable plate assembly 552 includes a through hole 554 that is contoured to the outer contours of the bone fixture 557. The through hole 554 thus forms a bone fixture interface section that is contoured to the exposed section of the bone fixture 557. In an example, the sections are sized and dimensioned such that at least a slip fit or an interference fit exists with respect to the sections. A plate screw 556 is used to secure plate assembly 552 to the bone fixture 557. The head of the plate screw 556 can be larger than the hole through the implantable plate assembly 552, and thus the plate screw 556 positively retains the implantable plate assembly 552 to the bone fixture 557. The portions of plate screw 556 that interface with the bone fixture 557 substantially correspond to an abutment screw detailed in greater detail below, thus permitting the plate screw 556 to readily fit into an existing bone fixture used in a percutaneous bone conduction device. In an example, the plate screw 556 is configured so that the same tools and procedures that are used to install and/or remove an abutment screw from the bone fixture 557 can be used to install and/or remove the plate screw 556 from the bone fixture 557. In some examples, there may be a silicone layer 559 disposed between the plate 555 and bone 538.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. For examples, while certain technologies described herein were primarily described in the context of auditory prostheses (e.g., cochlear implants), technologies disclosed herein are applicable to medical devices generally (e.g., medical devices providing pain management functionality or therapeutic electrical stimulation, such as deep brain stimulation). In general, additional configurations can be used to practice the processes and systems herein and/or some aspects described can be excluded without departing from the processes and systems disclosed herein. Further, the techniques described herein can be applicable to enhancing the delivery of other stimuli, such as visual stimuli, tactile stimuli, olfactory stimuli, taste stimuli, or another stimuli. Likewise, the devices used herein need not be limited to auditory prostheses and can be other medical devices configured to support a human sense, such as bionic eyes.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A method comprising:
obtaining a sound signal;
generating, based on the sound signal, cochlear stimulation configured to stimulate cochlear tissue; and
generating, based on the sound signal, vestibular stimulation configured to stimulate vestibular tissue.

2. The method of claim 1, wherein generating the vestibular stimulation includes generating electrical stimulation via one or more vestibular electrodes disposed proximate an otolith organ.

3. The method of claim 1, wherein generating the vestibular stimulation includes generating vibratory stimulation.

4. The method of claim 1, wherein the sound signal includes a set of frequency components, and wherein at least a portion of the vestibular stimulation and a portion of the cochlear stimulation are based on a same subset of the set of frequency components.

5. The method of claim 1, further comprising:
generating a vestibular optimized sound signal by optimizing the sound signal for delivery to target the vestibular tissue, and
generating the vestibular stimulation based on the vestibular optimized sound signal.

6. The method of claim 5, wherein optimizing the sound signal for delivery to target the vestibular tissue includes:
enhancing a fundamental frequency in a sound spectrum of the sound signal.

7. The method of claim 1,
wherein the cochlear stimulation is electrical stimulation.

8. An apparatus, comprising:
a cochlear stimulator configured to stimulate cochlear tissue;
a vestibular stimulator configured to stimulate vestibular tissue; and
a sound processor configured to:
generate cochlear stimulation with the cochlear stimulator based on a sound signal; and
generate vestibular stimulation with the vestibular stimulator based on the sound signal.

9. The apparatus of claim 8, wherein the cochlear stimulator comprises a plurality of electrodes.

10. The apparatus of claim 8, wherein the vestibular stimulator comprises a plurality of intravestibular electrodes.

11. The apparatus of claim 8, wherein the sound processor is further configured to:
identify a subset of frequency components of the sound signal, and to generate the vestibular stimulation based on the subset of frequency components.

12. The apparatus of claim 11, wherein the sound processor is configured to generate the vestibular stimulation without contribution of the frequency components outside of the subset.

13. The apparatus of claim 11, wherein the sound processor is configured to generate the vestibular stimulation such that the vestibular stimulation is perceived as a hearing percept rather than a balance percept.

14. The apparatus of claim 8,
wherein the vestibular stimulator is configured to be disposed proximate at least one of an otolith organ or an oval window of a recipient.

15. A method comprising:
obtaining a sound signal;
generating a vestibular-optimized sound signal based on the sound signal; and
generating, based on the vestibular-optimized sound signal, vestibular stimulation configured to cause a hearing precept in a recipient.

16. The method of claim 15, wherein generating the vestibular-optimized sound signal:
enhancing a fundamental frequency in a sound spectrum of the sound signal.

17. The method of claim 15, wherein generating the vestibular-optimized sound signal includes:
selecting a subset of a set of frequency components of the sound signal,
wherein the vestibular-optimized sound signal includes the subset and lacks frequency components outside the subset.

18. The method of claim 15, wherein generating the vestibular-optimized sound signal includes:
amplifying portions of the sound signal that are within a detection band of a vestibular organ.

19. The method of claim 15, wherein generating the vestibular-optimized sound signal includes:
tuning the sound signal to cause a beneficial balance percept.

20. The method of claim 15,
wherein the method further comprises electrically stimulating cochlear tissue based on the sound signal.
